SEQUENCE LISTING (148 sequences)

SEQ ID NO: 1 - protein, 9 aa, synthetic construct
GFAFSRYPM

SEQ ID NO: 2 - protein, 9 aa, synthetic construct
GYTYRWCNM

SEQ ID NO: 3 - protein, 9 aa, synthetic construct
GYTYSSYCM

SEQ ID NO: 4 - protein, 11 aa, synthetic construct
VYTISNPKKCM

SEQ ID NO: 5 - protein, 9 aa, synthetic construct
GYTYSAGSM

SEQ ID NO: 6 - protein, 9 aa, synthetic construct
GFTFSNYGM

SEQ ID NO: 7 - protein, 9 aa, synthetic construct
RFTYNNGWL

SEQ ID NO: 8 - protein, 9 aa, synthetic construct
TYTYKIYSM

SEQ ID NO: 9 - protein, 9 aa, synthetic construct
GFTFSNYGM

SEQ ID NO: 10 - protein, 9 aa, synthetic construct
GYISSSDYM

SEQ ID NO: 11 - protein, 9 aa, synthetic construct
GVTHTSNCM

SEQ ID NO: 12 - protein, 9 aa, synthetic construct
GVTHTSNCM

SEQ ID NO: 13 - protein, 9 aa, synthetic construct
GVTGRICTM

SEQ ID NO: 14 - protein, 9 aa, synthetic construct
GFTFSHYDM

SEQ ID NO: 15 - protein, 9 aa, synthetic construct
GDTYSRYIM

SEQ ID NO: 16 - protein, 9 aa, synthetic construct
KYSYSTDCM

SEQ ID NO: 17 - protein, 9 aa, synthetic construct
GSSDLYNWM

SEQ ID NO: 18 - protein, 9 aa, synthetic construct
MYGIRNLYM

SEQ ID NO: 19 - protein, 9 aa, synthetic construct
GFRFSSYGM

SEQ ID NO: 20 - protein, 9 aa, synthetic construct
EATYNDFCM

SEQ ID NO: 21 - protein, 9 aa, synthetic construct
KLTYCTYDM

SEQ ID NO: 22 - protein, 9 aa, synthetic construct
RDTSSRNCM

SEQ ID NO: 23 - protein, 8 aa, synthetic construct
SMSNINCM

SEQ ID NO: 24 - protein, 9 aa, synthetic construct
GYIDSDYCM

SEQ ID NO: 25 - protein, 9 aa, synthetic construct
GFTYVSYCM

SEQ ID NO: 26 - protein, 9 aa, synthetic construct
GLTDVSYIM

SEQ ID NO: 27 - protein, 9 aa, synthetic construct
GFTFRNYYM

SEQ ID NO: 28 - protein, 9 aa, synthetic construct
GFTFSNYWM

SEQ ID NO: 29 - protein, 9 aa, synthetic construct
GITYRVCSR

SEQ ID NO: 30 - protein, 9 aa, synthetic construct
GNTYTSRLM

SEQ ID NO: 31 - protein, 9 aa, synthetic construct
GYNYKTYRM

SEQ ID NO: 32 - protein, 9 aa, synthetic construct
GDTHRTYFI

SEQ ID NO: 33 - protein, 9 aa, synthetic construct
GYAYRPYCM

SEQ ID NO: 34 - protein, 10 aa, synthetic construct
SGITYSRYCM

SEQ ID NO: 35 - protein, 9 aa, synthetic construct
GFTISRNAM

SEQ ID NO: 36 - protein, 9 aa, synthetic construct
GYAFSTYSM

SEQ ID NO: 37 - protein, 9 aa, synthetic construct
GYTFKDYCM

SEQ ID NO: 38 - protein, 9 aa, synthetic construct
RYTYRTYCM

SEQ ID NO: 39 - protein, 9 aa, synthetic construct
GFFVGSHLM

SEQ ID NO: 40 - protein, 9 aa, synthetic construct
GYRYSTYCM

SEQ ID NO: 41 - protein, 9 aa, synthetic construct
GYRYSTYCM

SEQ ID NO: 42 - protein, 9 aa, synthetic construct
GYTFSSYCL

SEQ ID NO: 43 - protein, 9 aa, synthetic construct
EYTYSNYYM

SEQ ID NO: 44 - protein, 9 aa, synthetic construct
EYAYSNAYM

SEQ ID NO: 45 - protein, 13 aa, synthetic construct
GYYGYTYNNNMCM

SEQ ID NO: 46 - protein, 8 aa, synthetic construct
EYSYINCM

SEQ ID NO: 47 - protein, 9 aa, synthetic construct
GYTYRSYCM

SEQ ID NO: 48 - protein, 9 aa, synthetic construct
GYSYTSNFM

SEQ ID NO: 49 - protein, 11 aa, synthetic construct
STIFPGGDTPY

SEQ ID NO: 50 - protein, 10 aa, synthetic construct
SRVISDGTTA

SEQ ID NO: 51 - protein, 10 aa, synthetic construct
ATIDSYGYTS

SEQ ID NO: 52 - protein, 11 aa, synthetic construct
AGIHRGVGTTY

SEQ ID NO: 53 - protein, 11 aa, synthetic construct
AVDSGVDGITA

SEQ ID NO: 54 - protein, 11 aa, synthetic construct
SGIETGGGSTY

SEQ ID NO: 55 - protein, 11 aa, synthetic construct
AAIYTTAGSTF

SEQ ID NO: 56 - protein, 10 aa, synthetic construct
AAIDSDGSTI

SEQ ID NO: 57 - protein, 11 aa, synthetic construct
SGIETGGGSTY

SEQ ID NO: 58 - protein, 11 aa, synthetic construct
ATLFTGSTTTY

SEQ ID NO: 59 - protein, 14 aa, synthetic construct
ARIFSGAGGITNTY

SEQ ID NO: 60 - protein, 11 aa, synthetic construct
AFMHTADSTTR

SEQ ID NO: 61 - protein, 10 aa, synthetic construct
SRITRAGDTT

SEQ ID NO: 62 - protein, 11 aa, synthetic construct
SGINSDGGDTY

SEQ ID NO: 63 - protein, 10 aa, synthetic construct
ATISADGYTG

SEQ ID NO: 64 - protein, 10 aa, synthetic construct
ASIDSERGTS

SEQ ID NO: 65 - protein, 11 aa, synthetic construct
AAIYMRIERTH

SEQ ID NO: 66 - protein, 11 aa, synthetic construct
AAISPSAGGTY

SEQ ID NO: 67 - protein, 11 aa, synthetic construct
SGISNDGSEAY

SEQ ID NO: 68 - protein, 11 aa, synthetic construct
AAITTEGSYTY

SEQ ID NO: 69 - protein, 10 aa, synthetic construct
SGIDSTGSTS

SEQ ID NO: 70 - protein, 11 aa, synthetic construct
ASIRSGIGATN

SEQ ID NO: 71 - protein, 11 aa, synthetic construct
ALLYTRASGTI

SEQ ID NO: 72 - protein, 9 aa, synthetic construct
ATIANFKPI

SEQ ID NO: 73 - protein, 10 aa, synthetic construct
GTIDSTLSTK

SEQ ID NO: 74 - protein, 10 aa, synthetic construct
ACITDGGPTA

SEQ ID NO: 75 - protein, 11 aa, synthetic construct
SGIDIWGVTTD

SEQ ID NO: 76 - protein, 10 aa, synthetic construct
SSISTGGTTY

SEQ ID NO: 77 - protein, 10 aa, synthetic construct
SSISNDGVPA

SEQ ID NO: 78 - protein, 11 aa, synthetic construct
AAIGDYGTSIY

SEQ ID NO: 79 - protein, 10 aa, synthetic construct
AVIDSDGSTK

SEQ ID NO: 80 - protein, 10 aa, synthetic construct
ATMAESGVTG

SEQ ID NO: 81 - protein, 10 aa, synthetic construct
ATINIDGSTS

SEQ ID NO: 82 - protein, 10 aa, synthetic construct
ASIDTVGSTN

SEQ ID NO: 83 - protein, 12 aa, synthetic construct
SGIERGGGGPTY

SEQ ID NO: 84 - protein, 10 aa, synthetic construct
AVINKYGTTK

SEQ ID NO: 85 - protein, 10 aa, synthetic construct
AIISPYGGWD

SEQ ID NO: 86 - protein, 10 aa, synthetic construct
AAMNSDGNTV

SEQ ID NO: 87 - protein, 10 aa, synthetic construct
ASIGDTSTIG

SEQ ID NO: 88 - protein, 10 aa, synthetic construct
AAIDADGFTS

SEQ ID NO: 89 - protein, 11 aa, synthetic construct
ALINTRGDSTS

SEQ ID NO: 90 - protein, 10 aa, synthetic construct
VTICGSGTPT

SEQ ID NO: 91 - protein, 11 aa, synthetic construct
ACLNNGGISTY

SEQ ID NO: 92 - protein, 11 aa, synthetic construct
ATLYISIGRTK

SEQ ID NO: 93 - protein, 11 aa, synthetic construct
ARISLSSGTSI

SEQ ID NO: 94 - protein, 11 aa, synthetic construct
ACMYYPGGTTT

SEQ ID NO: 95 - protein, 10 aa, synthetic construct
AAIGSDGSTR

SEQ ID NO: 96 - protein, 11 aa, synthetic construct
AAIDTRGVRTF

SEQ ID NO: 97 - protein, 8 aa, synthetic construct
DGIGVSSP

SEQ ID NO: 98 - protein, 13 aa, synthetic construct
GLRTGDTQGCTNY

SEQ ID NO: 99 - protein, 20 aa, synthetic construct
SGRLQASGGYCYIHSHAYNY

SEQ ID NO: 100 - protein, 18 aa, synthetic construct
RFCLTDYGLGDFNRLYDY

SEQ ID NO: 101 - protein, 15 aa, synthetic construct
FRGWGLTLDRRRFNY

SEQ ID NO: 102 - protein, 8 aa, synthetic construct
GVVGISNP

SEQ ID NO: 103 - protein, 15 aa, synthetic construct
EKHSGATWAAGRYTY

SEQ ID NO: 104 - protein, 20 aa, synthetic construct
ETYGWEVAGPSALSSYEYNY

SEQ ID NO: 105 - protein, 8 aa, synthetic construct
GTIGISSP

SEQ ID NO: 106 - protein, 20 aa, synthetic construct
TTGALWWPAREALRNTNFAY

SEQ ID NO: 107 - protein, 17 aa, synthetic construct
TYVLYARSCRITNEYNY

SEQ ID NO: 108 - protein, 19 aa, synthetic construct
DSRYRPYYSSCPRSTDDLY

SEQ ID NO: 109 - protein, 10 aa, synthetic construct
RYGSFCTSNR

SEQ ID NO: 110 - protein, 8 aa, synthetic construct
GTIGISEP

SEQ ID NO: 111 - protein, 17 aa, synthetic construct
SRSNWYGLSLRRSDFAY

SEQ ID NO: 112 - protein, 19 aa, synthetic construct
HSWSTVCGGVDWSESAGNV

SEQ ID NO: 113 - protein, 22 aa, synthetic construct
AFLPRGIQASWATTLDPTMYPS

SEQ ID NO: 114 - protein, 16 aa, synthetic construct
DDWLHTGYLSPKEYHL

SEQ ID NO: 115 - protein, 8 aa, synthetic construct
GLVGVTTP

SEQ ID NO: 116 - protein, 18 aa, synthetic construct
KRFCYSRVRSLTSSEYSQ

SEQ ID NO: 117 - protein, 12 aa, synthetic construct
VLGGSACRGGPY

SEQ ID NO: 118 - protein, 16 aa, synthetic construct
GFCPYGPSLHLASYAY

SEQ ID NO: 119 - protein, 16 aa, synthetic construct
DSYNCHLGSWYHFTRY

SEQ ID NO: 120 - protein, 12 aa, synthetic construct
VSRCGVVPPNYH

SEQ ID NO: 121 - protein, 19 aa, synthetic construct
TDIRSSCWHASIRGPDFGY

SEQ ID NO: 122 - protein, 15 aa, synthetic construct
TRGYGFTLRSADYKY

SEQ ID NO: 123 - protein, 17 aa, synthetic construct
ETDPFSGGSWGGETGDY

SEQ ID NO: 124 - protein, 19 aa, synthetic construct
PRHVEGSSTGTSNFDERGK

SEQ ID NO: 125 - protein, 14 aa, synthetic construct
SCRRVEWTIRYDDY

SEQ ID NO: 126 - protein, 15 aa, synthetic construct
KRWGGIWSTVTDYNY

SEQ ID NO: 127 - protein, 18 aa, synthetic construct
DTAFVGEVVTLDQYEYNY

SEQ ID NO: 128 - protein, 15 aa, synthetic construct
AYGYGLSLRPHDYRY

SEQ ID NO: 129 - protein, 17 aa, synthetic construct
ETMGWDRCSLLGYEYNY

SEQ ID NO: 130 - protein, 18 aa, synthetic construct
EPGPYCSGGSTVTVGYEY

SEQ ID NO: 131 - protein, 7 aa, synthetic construct
GIGVSEP

SEQ ID NO: 132 - protein, 18 aa, synthetic construct
HLNLWIGRHVPPAEIFDY

SEQ ID NO: 133 - protein, 15 aa, synthetic construct
GPSYCSRAYIQTAPY

SEQ ID NO: 134 - protein, 17 aa, synthetic construct
ESYGFEIATLTPDEYNY

SEQ ID NO: 135 - protein, 17 aa, synthetic construct
AREGSWGWSRRERDFAY

SEQ ID NO: 136 - protein, 16 aa, synthetic construct
GCNFDGGSDIRYGLDY

SEQ ID NO: 137 - protein, 19 aa, synthetic construct
PLLPGGACSRTALVPDFRY

SEQ ID NO: 138 - protein, 14 aa, synthetic construct
SLRYYCSPRSDFTY

SEQ ID NO: 139 - protein, 15 aa, synthetic construct
STYLRGACRDYNYAY

SEQ ID NO: 140 - protein, 17 aa, synthetic construct
HRGSCWDETIFPANFES

SEQ ID NO: 141 - protein, 20 aa, synthetic construct
DLTQYARNCGRLLNFYESPS

SEQ ID NO: 142 - protein, 16 aa, synthetic construct
GVRIWGISCLHNEYRY

SEQ ID NO: 143 - protein, 20 aa, synthetic construct
DLAGIGDGDYCYPNVDEYNY

SEQ ID NO: 144 - protein, 15 aa, synthetic construct
GVGFGYSLSGKYYKY

SEQ ID NO: 145 - DNA, 23 bp, synthetic construct
  source 1..23: mol_type = other DNA
gtcctggctgctcttctacaagg

SEQ ID NO: 146 - DNA, 23 bp, synthetic construct
  source 1..23: mol_type = other DNA
ggtacgtgctgttgaactgttcc

SEQ ID NO: 147 - DNA, 65 bp, synthetic construct
  source 1..65: mol_type = other DNA
aacatgccatgactcgcggctcaaccggccatggctgakgtbcagctgcaggcgtctggrggagg

SEQ ID NO: 148 - DNA, 53 bp, synthetic construct
  source 1..53: mol_type = other DNA
gttattattattcagattattagtgcggccgctggagacggtgaccwgggtcc

(19) United States
(12) Patent Application Publication
(10) Pub. No.: US 2025/0092122 A1
(43) Pub. Date: Mar. 20, 2025

(54) SINGLE-DOMAIN ANTIBODY AGAINST CPV AND USES THEREOF

(71) Applicant: Lanzhou Veterinary Research Institute Chinese Academy of Agricultural Sciences, Lanzhou (CN)

(72) Inventors: Huichen GUO, Lanzhou (CN); Shiqi SUN, Lanzhou (CN); Suyu MU, Lanzhou (CN); Shuzhen TAN, Lanzhou (CN); Shuanghui YIN, Lanzhou (CN); Hu DONG, Lanzhou (CN); Yun ZHANG, Lanzhou (CN); Jianing WANG, Lanzhou (CN); Manyuan BAI, Lanzhou (CN); Jinen WU, Lanzhou (CN); Zhidong TENG, Lanzhou (CN)

(21) Appl. No.: 18/890,711

(22) Filed: Sep. 19, 2024

(30) Foreign Application Priority Data
Sep. 19, 2023 (CN) 202311204292.6

(51) Int. Cl. C07K 16/08

(52) U.S. Cl. CPC C07K 16/081; C07K 2317/24; C07K 2317/569; C07K 2317/76; C07K 2317/92

(57) ABSTRACT

A single-domain antibody or an antigen-binding fragment thereof for neutralizing canine parvovirus (CPV) including a heavy chain encompassing a variable region. The variable region includes three complementarity-determining regions (CDRs): CDR1, CDR2, and CDR3. The three complementarity-determining regions CDR1, CDR2, and CDR3 have a combination of different amino acid sequences. The antibodies effectively prevent CPV from infecting F81 cells, exhibiting high binding affinity and specificity, and significant neutralizing activity against CPV. Consequently, the antibody sequences and expression vectors of the disclosure are valuable tools for various applications in CPV epitope identification, prevention, treatment, and diagnosis.

### CROSS-REFERENCE TO RELATED APPLICATIONS

Pursuant to 35 U.S.C.§ 119 and the Paris Convention Treaty, this application claims foreign priority to Chinese Patent Application No. 202311204292.6 filed Sep. 19, 2023, the contents of which, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, MA 02142.

### INCORPORATION-BY-REFERENCE OF SEQUENCE LISTING

This application comprises a sequence listing, which has been submitted electronically in XML file and is incorporated herein by reference in its entirety. The XML file, created on Aug. 7, 2024, is named LZSY-01101-UUS.xml, and is 129,502 bytes in size.

### BACKGROUND

The disclosure relates to the field of biotechnology, and more particularly, to a single-domain antibody against CPV and uses thereof.

Canine parvovirus (CPV) is a non-enveloped, single-stranded DNA virus belonging to the Parvoviridae family and the Parvovirus genus. CPV has a diameter of 21-24 nm, with a genome size of approximately 5.2 kb, containing two open reading frames (ORFs) that encode structural proteins (VP1 and VP2) and nonstructural proteins (NS1 and NS2). CPV is a DNA virus that exhibits a very high mutation rate, approximately 104 per site per year, similar to the mutation rate of RNA viruses, resulting in rapid and continuous antigenic evolution and variation. Canine parvovirus type 1 (CPV-1), commonly referred to as minute virus of canines, is genetically unrelated to canine parvovirus type 2 (CPV-2), which is a major enteric pathogen causing gastroenteritis in dogs/puppies. CPV-2 is a non-enveloped virus with an icosahedral capsid enclosing a single-stranded DNA.

Within the icosahedral capsid, protein VP2 is a major capsid protein for determining the antigenicity of CPV. CPV appears to be a naturally derived host range variant of a virus that is similar to the feline panleukopenia virus (FPV). CPV-2 is an extremely lethal and contagious virus that can cause acute hemorrhagic diarrhea in dogs worldwide. Similar to other parvoviruses, CPV-2 causes a 100% morbidity rate and a mortality rate of up to 70% in pups, although the mortality rate is less than 1% in adult dogs. Treatment for CPV infection includes supportive care, such as drug therapy, antibiotics, antiemetics, anti-viral treatment, supportive treatments, and dietary management.

CPV-2a, CPV-2b, and CPV-2c are the three main subtypes of canine parvovirus. In the early 1980s, CPV-2a was identified as the predominant parvovirus in dogs. In 1984, the new subtype CPV-2b first appeared in the United States and then spread worldwide, causing severe gastroenteritis and myocarditis in dogs, cats, and wild carnivores. CPV-2c is the third variant of CPV-2, first discovered in Italy in 2000, and quickly spread to Europe, Americas, Asia, and Australia. CPV-2c has been reported to be the primary variant causing canine parvovirus disease globally. The three variants CPV-2a, CPV-2b, and CPV-2c are typed based on an amino acid change in the 426^{th} amino acid residue. In CPV-2a, the amino acid residue is asparagine. In CPV-2b, the amino acid residue is aspartic acid. In CPV-2c, the amino acid residue is glutamic acid.

CPV is highly resilient, capable of surviving at 4-10° C. for six months, at 37° C. for 14 days, at 56° C. for 24-36 hours, and at 60° C. for 1 hour. CPV can persist in feces for several months to years and still cause infections. CPV is highly contagious and can infect all canines at any age, though dogs aged 1-6 months are most susceptible. Clinically, canine parvovirus disease primarily presents as enteritis, myocarditis, or a mixed form thereof. The enteritis form commonly affects pups aged 2-6 months, with symptoms including lethargy, loss of appetite, depression, vomiting, and diarrhea, often resulting in a high mortality rate. The myocarditis form is common in puppies aged 4-6 weeks, with no obvious symptoms, sudden onset, rapid death, and a mortality rate of 60-100%.

Vaccination of susceptible animals to induce specific immunity is the best method for preventing CPV-2 infection, but most vaccines on the market are prepared with attenuated CPV-2 strains. The attenuated vaccines carry risks, including strain mutation and reversion to virulence, and may be affected by maternal antibodies leading to potential immune failure. As CPV-2 has evolved into various strains such as CPV-2a, CPV-2b, and CPV-2c, the effectiveness of existing vaccines has decreased, highlighting the urgent need for new or improved treatment options.

Currently, treatment for CPV mainly involves the use of antiviral drugs with high titers of convalescent serum. Despite the treatment, many cases still result in death. Antibody-based treatments present a promising alternative. Neutralizing monoclonal antibodies (nMAbs) can be developed to specifically target and neutralize CPV. Techniques in structural biology can identify the neutralizing sites on CPV, which facilitates the creation of broad-spectrum therapeutics that can be effective against various CPV subtypes. Single-domain antibodies (VHHs) are derived from camelid antibodies that lack light chains and offer several advantages for biotechnological applications, such as high production levels and stability. VHHs can be easily selected from phage display libraries and produced in polyvalent forms to enhance antibody-antigen affinity, or to create bispecific antibodies that bind two unrelated antigens.

Currently, several mouse-derived monoclonal antibodies (mAbs) have been reported to inhibit the replication of CPV through hybridoma technology. For example, Yin et al. have screened a monoclonal antibody targeting VP2, which can inhibit CPV at concentrations as low as 0.5-1 μg. Additionally, patents by Zhao et al. have developed dog- and cat-derived monoclonal antibodies for the prevention and/or treatment of CPV infections in animals such as dogs and cats. However, conventional antibodies face challenges in recombinant expression. Meanwhile, Jia et al. have obtained CPV-2c-specific nanobodies through phage display technology (with a library capacity of 2×10⁶ CFU/mL), but the nanobodies lack neutralizing activity.

### SUMMARY

To solve the aforesaid problems, the first objective of the disclosure is to provide a single-domain antibody or an antigen-binding fragment thereof for neutralizing canine parvovirus (CPV). The single-domain antibody or antigen-binding fragment thereof comprises a heavy chain encompassing a variable region. The variable region comprises three complementarity-determining regions (CDRs): CDR1, CDR2, and CDR3. The three complementarity-determining regions CDR1, CDR2, and CDR3 have a combination of amino acid sequences as follows:
(1) CDR1 shown in SEQ ID NO: 1, CDR2 shown in SEQ ID NO: 49, and CDR3 shown in SEQ ID NO: 97;
(2) CDR1 shown in SEQ ID NO: 2, CDR2 shown in SEQ ID NO: 50, and CDR3 shown in SEQ ID NO: 98;
(3) CDR1 shown in SEQ ID NO: 3, CDR2 shown in SEQ ID NO: 51, and CDR3 shown in SEQ ID NO: 99;
(4) CDR1 shown in SEQ ID NO: 4, CDR2 shown in SEQ ID NO: 52, and CDR3 shown in SEQ ID NO: 100;
(5) CDR1 shown in SEQ ID NO: 5, CDR2 shown in SEQ ID NO: 53, and CDR3 shown in SEQ ID NO: 101;
(6) CDR1 shown in SEQ ID NO: 6, CDR2 shown in SEQ ID NO: 54, and CDR3 shown in SEQ ID NO: 102;
(7) CDR1 shown in SEQ ID NO: 7, CDR2 shown in SEQ ID NO: 55, and CDR3 shown in SEQ ID NO: 103;
(9) CDR1 shown in SEQ ID NO: 8, CDR2 shown in SEQ ID NO: 56, and CDR3 shown in SEQ ID NO: 104;
(10) CDR1 shown in SEQ ID NO: 9, CDR2 shown in SEQ ID NO: 57, and CDR3 shown in SEQ ID NO: 105;
(12) CDR1 shown in SEQ ID NO: 10, CDR2 shown in SEQ ID NO: 58, and CDR3 shown in SEQ ID NO: 106;
(13) CDR1 shown in SEQ ID NO: 11, CDR2 shown in SEQ ID NO: 59, and CDR3 shown in SEQ ID NO: 107;
(14) CDR1 shown in SEQ ID NO: 12, CDR2 shown in SEQ ID NO: 60, and CDR3 shown in SEQ ID NO: 108;
(15) CDR1 shown in SEQ ID NO: 13, CDR2 shown in SEQ ID NO: 61, and CDR3 shown in SEQ ID NO: 109;
(17) CDR1 shown in SEQ ID NO: 14, CDR2 shown in SEQ ID NO: 62, and CDR3 shown in SEQ ID NO: 110;
(18) CDR1 shown in SEQ ID NO: 15, CDR2 shown in SEQ ID NO: 63, and CDR3 shown in SEQ ID NO: 111;
(19) CDR1 shown in SEQ ID NO: 16, CDR2 shown in SEQ ID NO: 64, and CDR3 shown in SEQ ID NO: 112;
(20) CDR1 shown in SEQ ID NO: 17, CDR2 shown in SEQ ID NO: 65, and CDR3 shown in SEQ ID NO: 113;
(21) CDR1 shown in SEQ ID NO: 18, CDR2 shown in SEQ ID NO: 66, and CDR3 shown in SEQ ID NO: 114;
(22) CDR1 shown in SEQ ID NO: 19, CDR2 shown in SEQ ID NO: 67, and CDR3 shown in SEQ ID NO: 115;
(24) CDR1 shown in SEQ ID NO: 20, CDR2 shown in SEQ ID NO: 68, and CDR3 shown in SEQ ID NO: 116;
(25) CDR1 shown in SEQ ID NO: 21, CDR2 shown in SEQ ID NO: 69, and CDR3 shown in SEQ ID NO: 117;
(26) CDR1 shown in SEQ ID NO: 22, CDR2 shown in SEQ ID NO: 70, and CDR3 shown in SEQ ID NO: 118;
(27) CDR1 shown in SEQ ID NO: 23, CDR2 shown in SEQ ID NO: 71, and CDR3 shown in SEQ ID NO: 119;
(29) CDR1 shown in SEQ ID NO: 24, CDR2 shown in SEQ ID NO: 72, and CDR3 shown in SEQ ID NO: 120;
(30) CDR1 shown in SEQ ID NO: 25, CDR2 shown in SEQ ID NO: 73, and CDR3 shown in SEQ ID NO: 121;
(31) CDR1 shown in SEQ ID NO: 26, CDR2 shown in SEQ ID NO: 74, and CDR3 shown in SEQ ID NO: 122;
(32) CDR1 shown in SEQ ID NO: 27, CDR2 shown in SEQ ID NO: 75, and CDR3 shown in SEQ ID NO: 123;
(33) CDR1 shown in SEQ ID NO: 28, CDR2 shown in SEQ ID NO: 76, and CDR3 shown in SEQ ID NO: 124;
(34) CDR1 shown in SEQ ID NO: 29, CDR2 shown in SEQ ID NO: 77, and CDR3 shown in SEQ ID NO: 125;
(35) CDR1 shown in SEQ ID NO: 30, CDR2 shown in SEQ ID NO: 78, and CDR3 shown in SEQ ID NO: 126;
(36) CDR1 shown in SEQ ID NO: 31, CDR2 shown in SEQ ID NO: 79, and CDR3 shown in SEQ ID NO: 127;
(39) CDR1 shown in SEQ ID NO: 32, CDR2 shown in SEQ ID NO: 80, and CDR3 shown in SEQ ID NO: 128;
(40) CDR1 shown in SEQ ID NO: 33, CDR2 shown in SEQ ID NO: 81, and CDR3 shown in SEQ ID NO: 129;
(42) CDR1 shown in SEQ ID NO: 34, CDR2 shown in SEQ ID NO: 82, and CDR3 shown in SEQ ID NO: 130;
(43) CDR1 shown in SEQ ID NO: 35, CDR2 shown in SEQ ID NO: 83, and CDR3 shown in SEQ ID NO: 131;
(44) CDR1 shown in SEQ ID NO: 36, CDR2 shown in SEQ ID NO: 84, and CDR3 shown in SEQ ID NO: 132;
(45) CDR1 shown in SEQ ID NO: 37, CDR2 shown in SEQ ID NO: 85, and CDR3 shown in SEQ ID NO: 133;
(46) CDR1 shown in SEQ ID NO: 38, CDR2 shown in SEQ ID NO: 86, and CDR3 shown in SEQ ID NO: 134;
(47) CDR1 shown in SEQ ID NO: 39, CDR2 shown in SEQ ID NO: 87, and CDR3 shown in SEQ ID NO: 135;
(48) CDR1 shown in SEQ ID NO: 40, CDR2 shown in SEQ ID NO: 88, and CDR3 shown in SEQ ID NO: 136;
(49) CDR1 shown in SEQ ID NO: 41, CDR2 shown in SEQ ID NO: 89, and CDR3 shown in SEQ ID NO: 137;
(50) CDR1 shown in SEQ ID NO: 42, CDR2 shown in SEQ ID NO: 90, and CDR3 shown in SEQ ID NO: 138;
(51) CDR1 shown in SEQ ID NO: 43, CDR2 shown in SEQ ID NO: 91, and CDR3 shown in SEQ ID NO: 139;
(52) CDR1 shown in SEQ ID NO: 44, CDR2 shown in SEQ ID NO: 92, and CDR3 shown in SEQ ID NO: 140;
(53) CDR1 shown in SEQ ID NO: 45, CDR2 shown in SEQ ID NO: 93, and CDR3 shown in SEQ ID NO: 141;
(54) CDR1 shown in SEQ ID NO: 46, CDR2 shown in SEQ ID NO: 94, and CDR3 shown in SEQ ID NO: 142;
(55) CDR1 shown in SEQ ID NO: 47, CDR2 shown in SEQ ID NO: 95, and CDR3 shown in SEQ ID NO: 143; or
(56) CDR1 shown in SEQ ID NO: 48, CDR2 shown in SEQ ID NO: 96, and CDR3 shown in SEQ ID NO: 144.

In a class of this embodiment, the single-domain antibody or antigen-binding fragment thereof comprises:
(2) CDR1 shown in SEQ ID NO: 2, CDR2 shown in SEQ ID NO: 50, and CDR3 shown in SEQ ID NO: 98;
(10) CDR1 shown in SEQ ID NO: 9, CDR2 shown in SEQ ID NO: 57, and CDR3 shown in SEQ ID NO: 105;
(12) CDR1 shown in SEQ ID NO: 10, CDR2 shown in SEQ ID NO: 58, and CDR3 shown in SEQ ID NO: 106;
(42) CDR1 shown in SEQ ID NO: 34, CDR2 shown in SEQ ID NO: 82, and CDR3 shown in SEQ ID NO: 130;
(43) CDR1 shown in SEQ ID NO: 35, CDR2 shown in SEQ ID NO: 83, and CDR3 shown in SEQ ID NO: 131;
(45) CDR1 shown in SEQ ID NO: 37, CDR2 shown in SEQ ID NO: 85, and CDR3 shown in SEQ ID NO: 133;
(47) CDR1 shown in SEQ ID NO: 39, CDR2 shown in SEQ ID NO: 87, and CDR3 shown in SEQ ID NO: 135;
(48) CDR1 shown in SEQ ID NO: 40, CDR2 shown in SEQ ID NO: 88, and CDR3 shown in SEQ ID NO: 136; or
(55) CDR1 shown in SEQ ID NO: 47, CDR2 shown in SEQ ID NO: 95, and CDR3 shown in SEQ ID NO: 143.

In a class of this embodiment, the single-domain antibody or antigen-binding fragment thereof comprises:
(12) CDR1 shown in SEQ ID NO: 10, CDR2 shown in SEQ ID NO: 58, and CDR3 shown in SEQ ID NO: 106;
(42) CDR1 shown in SEQ ID NO: 34, CDR2 shown in SEQ ID NO: 82, and CDR3 shown in SEQ ID NO: 130;
(43) CDR1 shown in SEQ ID NO: 35, CDR2 shown in SEQ ID NO: 83, and CDR3 shown in SEQ ID NO: 131; or
(47) CDR1 shown in SEQ ID NO: 39, CDR2 shown in SEQ ID NO: 87, and CDR3 shown in SEQ ID NO: 135.

In a class of this embodiment, CDR1 has a sequence represented by one of SEQ ID NO: 1 to SEQ ID NO: 48, an amino acid sequence having at least 80%, 85%, 90%, 91%, 92%, 93%, 94%, 95%, 96%, 97%, 98%, or 99% sequence identity to one of SEQ ID NO: 1 to SEQ ID NO: 48, or a sequence variant having no more than 8 amino acid substitutions from one of SEQ ID NO: 1 to SEQ ID NO: 48; CDR2 has a sequence represented by one of SEQ ID NO: 49 to SEQ ID NO: 96, an amino acid sequence having at least 80%, 85%, 90%, 91%, 92%, 93%, 94%, 95%, 96%, 97%, 98%, or 99% sequence identity to one of SEQ ID NO: 49 to SEQ ID NO: 96, or a sequence variant having no more than 8 amino acid substitutions from one of SEQ ID NO: 49 to SEQ ID NO: 96; and CDR3 has a sequence represented by one of SEQ ID NO: 97 to SEQ ID NO: 144, an amino acid sequence having at least 80%, 85%, 90%, 91%, 92%, 93%, 94%, 95%, 96%, 97%, 98%, or 99% sequence identity to one of SEQ ID NO: 97 to SEQ ID NO: 144, or a sequence variant having no more than 8 amino acid substitutions from one of SEQ ID NO: 97 to SEQ ID NO: 144.

In a class of this embodiment, the antigen-binding fragment is selected from, but is not limited to, Fab, Fab′, F(ab′)2, Fv, single-chain Fv (scFv), single-chain Fab, diabody, single-domain antibody or sdAb (nanobody), camelid Ig, IgNAR, F(ab)′3 fragment, bispecific scFv, (scFv)2, mini antibody, bispecific antibody, trispecific antibody, tetraspecific antibody, and disulfide-stabilized Fv protein (dsFv).

In a class of this embodiment, the heavy chain of the antigen-binding fragment further comprises a constant region selected from porcine IgG, human IgG, and/or chicken IgY

In a class of this embodiment, the constant region IgG is IgG1, IgG2a, IgG2b, and/or IgG4.

In a class of this embodiment, the CPV is isolated from CPV-2a strain.

The second objective of the disclosure is to provide a nucleic acid molecule encoding the single-domain antibody or antigen-binding fragment thereof.

The third objective of the disclosure is to provide a recombinant vector or host cell comprising the nucleic acid molecule.

In a class of this embodiment, the recombinant vector is selected from DNA, RNA, viral vectors, transposons, other gene transfer systems, or a combination thereof.

In a class of this embodiment, the viral vector includes lentivirus, adenovirus, adeno-associated virus (AAV), retrovirus, or a combination thereof.

In a class of this embodiment, the host cell is a prokaryotic cell or eukaryotic cell.

In a class of this embodiment, the host cell includes, but is not limited to, Escherichia coli, yeast cells, mammalian cells, bacteriophages, or a combination thereof.

In a class of this embodiment, the prokaryotic cell includes, but is not limited to, Escherichia coli, Bacillus subtilis, Lactobacillus, Streptomyces, Proteus mirabilis, or a combination thereof.

In a class of this embodiment, the eukaryotic cell includes, but is not limited to, Pichia pastoris, Saccharomyces cerevisiae, Schizosaccharomyces pombe, Trichoderma, insect cells such as Spodoptera frugiperda, plant cells such as tobacco, BHK cells, CHO cells, COS cells, myeloma cells, or a combination thereof.

The fourth objective of the disclosure is to provide a chimeric antibody comprising the single-domain antibody or antigen-binding fragment thereof.

The fifth objective of the disclosure is to provide a fusion or conjugate comprising the single-domain antibody or antigen-binding fragment thereof, and the fusion or conjugate is formed by attaching the single-domain antibody or antigen-binding fragment thereof to a heterologous molecule.

In a class of this embodiment, the fusion or conjugate is formed by attaching the polypeptide of the single-domain antibody or antigen-binding fragment thereof against CPV to one or more heterologous molecules; the one or more heterologous molecules include, but not limited to proteins, peptides, markers, drugs, and cytotoxic agents.

The sixth objective of the disclosure is to provide a composition comprising the single-domain antibody or antigen-binding fragment thereof, the nucleic acid molecule, the recombinant vector or host cell, the chimeric antibody, and/or the fusion or conjugate.

The seventh objective of the disclosure is to provide a method for diagnosis, prevention, and/or treatment of viral infections comprising administering a patient in need thereof the single-domain antibody or antigen-binding fragment thereof, the nucleic acid molecule, the recombinant vector or host cell, the chimeric antibody, and/or the composition.

In a class of this embodiment, the viral infections are caused by CPV.

The eighth objective of the disclosure is to provide a kit for detecting CPV. The kit comprises an instruction manual and a reagent for detecting the single-domain antibody or antigen-binding fragment thereof, the nucleic acid molecule, the recombinant vector or host cell, the chimeric antibody, the fusion or conjugate, and/or the composition.

The following advantages are associated with the single-domain antibody or an antigen-binding fragment thereof of the disclosure.

The disclosed single-domain antibodies (nanobodies) have a low risk of triggering an immune response. The antibodies effectively prevent CPV from infecting F81 cells, exhibiting high binding affinity and specificity, and significant neutralizing activity against CPV. Consequently, the antibody sequences and expression vectors of the disclosure are valuable tools for various applications in CPV epitope identification, prevention, treatment, and diagnosis.

### BRIEF DESCRIPTION OF THE DRAWINGS

FIG. 1 shows the expression and purification of CPV antigen according to one example of the disclosure;

FIG. 2 shows the detection results of specific antibodies and neutralizing antibodies 4 weeks after immunizing a camel with antigen using Freund's adjuvant, according to one example of the disclosure;

FIG. 3 is an electrophoresis image showing the amplification of VHH sequences through nested PCR (first round of PCR), according to one example of the disclosure;

FIG. 4 is an electrophoresis image showing the amplification of VHH sequences through nested PCR (second round of PCR), according to one example of the disclosure;

FIG. 5 shows the sequences of 56 VHH sequences according to one example of the disclosure;

FIG. 6 shows a vector map of pNFCG1-EB containing Fc-VHH according to one example of the disclosure;

FIG. 7 shows the results of an ELISA assay for evaluating the binding affinity of VHH antibodies against CPV, according to one example of the disclosure; and

FIG. 8 shows the neutralizing activity of VHH antibodies according to one example of the disclosure.

### DETAILED DESCRIPTION

To further illustrate the disclosure, embodiments detailing a single-domain antibody against CPV and uses thereof are described below. It should be noted that the following embodiments are intended to describe and not to limit the disclosure.

The term “antibody”, as used herein, refers to a polypeptide that include at least the variable region of the light chain or the heavy chain of an immunoglobulin. The variable region specifically recognizes and binds to an antigen. The term encompasses a variety of antibody structures, including, but not limited to, monoclonal antibodies, polyclonal antibodies, single-chain antibodies, multi-chain antibodies, monospecific antibodies, multispecific antibodies (e.g., bispecific antibodies), fully human antibodies, chimeric antibodies, humanized antibodies, full-length antibodies, and antibody fragments, as long as the antibody exhibits the desired antigen-binding activity.

The term “antigen-binding fragment”, as described herein, refers to a portion or segment of a whole antibody that has fewer amino acid residues than the whole antibody but can still bind to an antigen or compete with the whole antibody (i.e., the antibody from which the antigen-binding fragment is derived) for binding. The antigen-binding fragment can be prepared through recombinant DNA technology or by enzymatic or chemical cleavage of the complete antibody. The antigen-binding fragment includes, but is not limited to, Fab, Fab′, F(ab′)2, Fv, single-chain Fv (scFv), single-chain Fab, diabody, single-domain antibody or nanobody (sdAb), camelid Ig, IgNAR, F(ab′)3 fragment, double scFv, (scFv)2, mini antibody, bispecific antibodies, trispecific antibody, tetraspecific antibody, and disulfide-stabilized Fv proteins (dsFv). The term also includes genetically engineered forms, such as chimeric antibody (e.g., humanized mouse antibody), hybrid antibody, and antigen-binding fragments thereof.

The term “variant”, as described herein, refers to antibodies that have been modified by 1-20 amino acid substitutions, deletions, and/or insertions in the target antibody region (such as the variable region of the heavy chain or the light chain, or the CDR regions of the heavy chain or the light chain), while substantially retaining the biological properties of the unmodified antibody. In one aspect, the disclosure provides variants of the antibodies described herein. In one example, the antibody variant retains at least 60%, 70%, 80%, 90%, or 100% of the biological activity (such as antigen-binding capacity or neutralizing capacity) of the unmodified antibody. The modifications can be applied individually or in combination to the variable region of the heavy chain, the variable region of the light chain, or each CDR region.

The term “chimeric antibody”, as described herein, refers to a recombinant protein that incorporates variable regions, including CDRs, derived from an antibody from one species, while the constant regions are sourced from a human antibody. Alternatively, the constant regions of the chimeric antibody may be obtained from other species, such as cats or dogs.

The term “isolated”, as described herein, refers to a substance or component that has been obtained through artificial means, separated from its natural state. The term does not imply absolute purity. Instead, the term acknowledges that there may be other impurities present in the isolated substance, provided these impurities do not affect the activity or function of the primary substance.

The term “sequence identity”, as used herein, refers to the degree of similarity between two or more biological sequences. The similarity is assessed within a sliding window that compares individual nucleotides in nucleic acids or amino acids in proteins. The “percent sequence identity” can be calculated as follows: aligning the two sequences within the sliding window to achieve the optimal alignment; identifying positions where nucleotide bases (e.g., A, T, C, G, I) or amino acid residues (e.g., Ala, Pro, Ser, Thr, Gly, Val, Leu, Ile, Phe, Tyr, Trp, Lys, Arg, His, Asp, Glu, Asn, Gln, Cys, and Met) are identical in both sequences; counting a total number of matching positions; dividing the total number of matching positions by a total number of positions in the sliding window (i.e., the size of the sliding window); and multiplying the result by 100 to obtain the percent sequence identity. The optimal alignment for determining the percent sequence identity can be achieved in various methods and software tools, such as BLAST, BLAST-2, ALIGN, or Megalign (DNASTAR) software. Those skilled in related art can adjust parameters and select suitable algorithms to achieve the optimal alignment over the full-length sequence being compared or within specific sequence regions of interest.

The term “vector”, as used herein, refers to a construct capable of introducing one or more genes or sequences of interest into a host cell and, preferably, to facilitate the expression of the genes or sequences within the host cell. The vectors include, but are not limited to, viral vectors, naked DNA or RNA expression vectors, plasmids, cosmids, or bacteriophage vectors, DNA or RNA expression vectors combined with cationic condensing agents, DNA or RNA expression vectors encapsulated in liposomes, and certain eukaryotic cells, such as production cells.

The terms “host cell”, as used herein, are used interchangeably to refer to cells that have been introduced with exogenous nucleic acids, including progeny of the cells. Host cells include “transformants” and “transformed cells,” which encompass primary transformed cells and progeny derived therefrom, regardless of the number of passages. Progeny may not be identical in nucleic acid content to the parent cell and may contain mutations. Cells that exhibit the same function or biological activity as those screened or selected from initially transformed cells are included.

The terms “neutralizing antibodies”, as used herein, refer to antibodies that bind to or interact with a target antigen and prevent the target antigen from binding to a binding partner, such as a receptor, thereby inhibiting or blocking the biological response that would result from the interaction of the target antigen with the binding partner.

The experimental methods and materials mentioned in the following examples are conventional and can be obtained through conventional commercial sources unless specified otherwise.

### Example 1 Preparation of VHH Antibody Against CPV

### 1.1 Construction of a Phage Display Library

Two healthy, appropriately aged camels, designated P-1 #and P-2 #, were selected for the immunization process. The two camels were immunized four times with an inactivated CPV (canine parvovirus) antigen. After the fourth immunization, 200 mL of peripheral blood was collected from each camel. Peripheral blood mononuclear cells (PBMCs) were isolated from the peripheral blood. RNA was extracted from the PBMCs and used to construct a camel VHH antibody library. The camel VHH antibody library had a capacity of 1.3×10⁹ unique VHH antibodies and a cloning positivity rate of 90%, with no contamination from aggressive bacteriophages.

### 1.1.1 Proliferation, Inactivation, and Purification of CPV Antigen

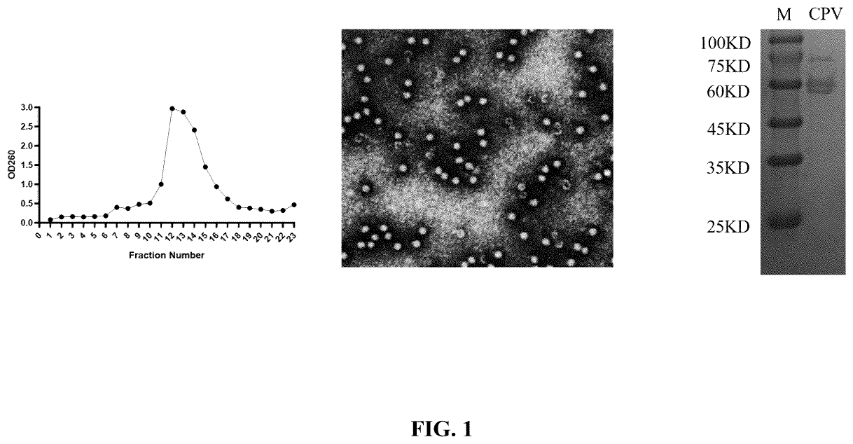

CPV-2a strain was cultured in F81 cells and inactivated using BEI (binary ethyleneimine) for 28 hours at 30° C. After incubation, the cell culture was centrifuged at 4000×g for 30 minutes to remove cell debris. The supernatant was then concentrated 1000-fold using a 100 kDa membrane, and a concentrated CPV-2a particles were obtained. A 10-50% sucrose density gradient was prepared. The concentrated CPV-2a particles were ultra-centrifugated at 250,000×g for 2 hours to form a CPV-2a band. The CPV-2a band was collected and dialyzed into PBS. The purified CPV-2a particles were observed using transmission electron microscopy (the results were shown in FIG. 1).

### 1.1.2 Camel Immunization and PBMC Separation

400 μg of SVA antigen was mixed with an equal volume of Freund's adjuvant and thoroughly emulsified; the resulting emulsion was administered subcutaneously at multiple sites along the neck of each camel; the initial immunization employed Freund's complete adjuvant; for subsequent booster immunizations, Freund's incomplete adjuvant was used; and the immunizations were administered at 14-day intervals.

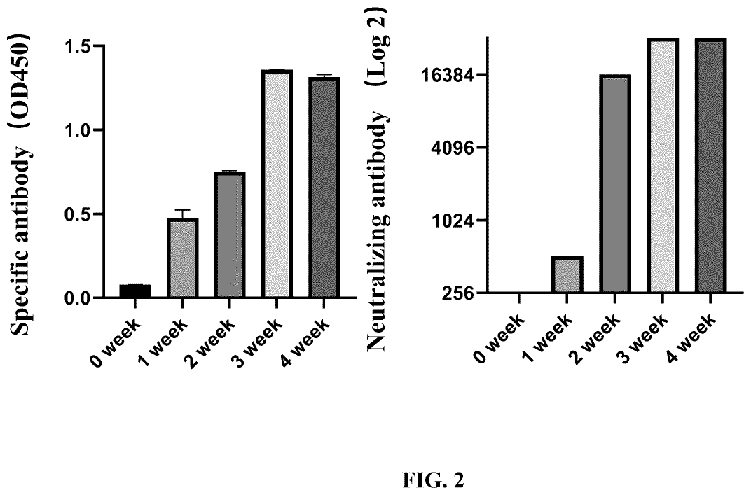

After the 2nd, 3rd, and 4th immunizations, a small amount of serum was collected and tested for antibody titers using ELISA array and neutralization assay. The results of antibody titers are depicted in FIG. 2, showing that the serum from camel P-1 #achieved a titer of 1:640,000, while the serum from camel P-2 #reached a titer of 1:2,560,000. The two antibody titer values indicate a strong immune response and satisfactory antibody production in both camels. After confirming satisfactory antibody titers, 200 mL of peripheral blood was collected from each camel. PBMCs were isolated using the Ficoll density gradient centrifugation method. The isolated PBMCs from the two camels were combined to construct the camel VHH antibody library.

As shown in FIG. 2, the specificity and neutralizing antibody levels were stable by the 3rd and 4th weeks.

### 1.1.3 RNA Extraction

An appropriate amount of Trizol was added to the isolated PBMCs to ensure thorough cell lysis. The lysate was centrifuged at 13000 rpm. The precipitate was discarded, and 200 μL of chloroform was added to each 1 mL of supernatant from the lysate. The resulting mixture was vigorously shaken for 30 seconds, incubated on ice for 5 minutes, and centrifuged at 13000 rpm for 10 minutes. After centrifugation, the solution was separated into three layers. 350-500 μL of the upper aqueous phase was carefully pipetted out, transferred into a centrifugal tube, mixed with an equal volume of isopropanol, and incubated at −20° C. for 30 minutes. After centrifugation at 13000 rpm for 10 minutes, the supernatant was discarded. The RNA pellet was washed twice with 1 mL of 75% cold ethanol and air-dried. DEPC water was treated with RRI (RNase Inhibitor) according to the amount of the RNA pellet. The dried RNA pellet was dissolved in the pre-treated DEPC water.

1.1.4 Insertion of Exogenous Genes into Phage Vectors

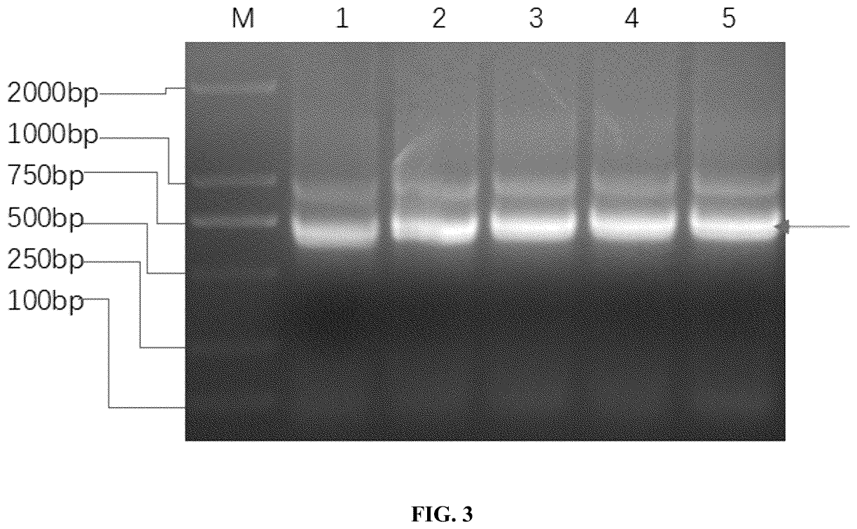

The isolated RNA was reverse transcribed into cDNA via RT-PCR using oligo-dT primers. In the first round of PCR, the cDNA was used as the template along with two primers: an upstream primer (SEQ ID NO: 145: GTC.CTG.GCT.GCT.CTT.CTA.CAA.GG) that binds to the variable region of the heavy chain of the VHH antibody and a downstream primer (SEQ ID NO: 146: GGT.ACG.TGC.TGT.TGA.ACT.GTT.CC) that binds to the constant region (CH2) of heavy chain of the VHH antibody. As shown in FIG. 3, the first round of PCR reaction yielded a 600 bp DNA band. The DNA band was used as a template in the second round of PCR.

Restriction sites were added to two VHH-specific primers used in the second round of PCR to ensure that the VHH gene can be easily inserted into the vector: the second round of PCR employed the two VHH-specific primers:

a forward primer with a restriction site:
SEQ ID NO: 147:
AACATGCCATGACTCGCGGCTCAACCGGCCATGGCTGAKGTBCAGCTGCA
GGCGTCTGGRGGAGG;
and
a reverse primer with a restriction site:
SEQ ID NO: 148:
GTTATTATTATTCAGATTATTAGTGCGGCCGCTGGAGACGGTGACCWGGG
TCC

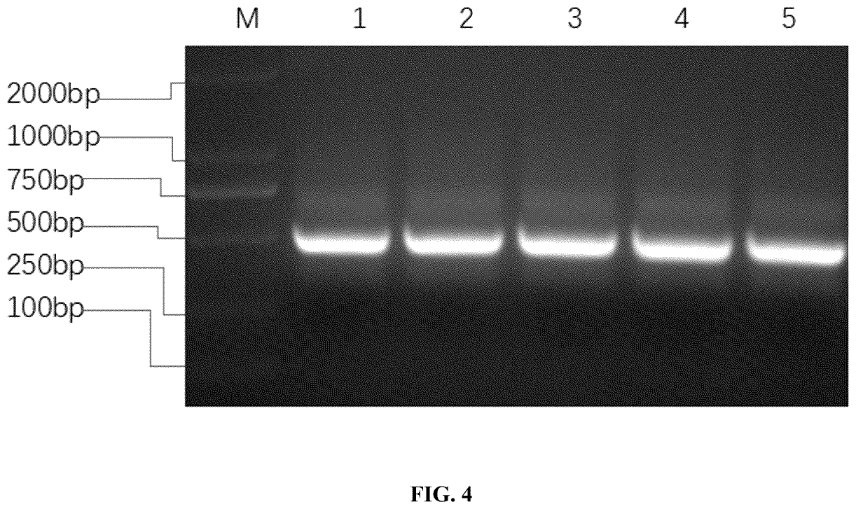

As shown in FIG. 4, the second round of PCR reaction yielded a VHH antibody gene.

The second round of PCR reaction yielded a VHH gene; a vector used for phage display was selected and digested with the same restriction enzymes that correspond to the restriction sites on the two specific primers; the VHH gene were also digested with the same restriction enzymes and ligated into the pre-digested vector using T4 DNA ligase; the ligation product was precipitated with ethanol to remove salts; and the purified ligation product was introduced into TG1 phage component cells via electroporation to construct a phage-displayed VHH antibody library with a capacity of 1.3×10⁹ different clones as shown in Table 1.

TABLE 1
A phage-displayed VHH antibody library

| Detection Plate Number | 1 | Total |
|---|---|---|
| Number of Competent | 5 | 5 |
| Cells | | |
| Number of Clones | 1320 | 1320 |

| | |
|---|---|
| Library Capacity | 5 × 200 × 10³ × (1320/1) = 1.3 × 10⁹ |
| Calculation | |

### 1.2 Screening of VHH Antibodies Against CPV

1.2.1 Selecting of Phages with Binding Affinity to CPV

1. Library preparation: 1.00×10¹³ pfu (plaque-forming units) of the phage-displayed VHH antibody library were used. A target protein was coated on a solid support at a concentration of 10 μg/mL. Before screening, the background from a blank plate and a control plate coated with 10 μg/mL of S protein was subtracted. The unbound phages were washed using 0.1% PBST, and then the bound phages were eluted from the solid support using Trypsin. The target enrichment rate was 1.04×10⁵.

2. First round of selecting: 6.00×10¹² pfu of the phage-displayed VHH antibody library were used. The target protein was coated on a solid support at a concentration of 5 μg/mL. Before screening, the background from a normal plate and a control plate coated with 5 μg/mL of S protein was subtracted. The unbound phages were washed using 0.2% PBST and then eluted using from the solid support using Trypsin. The target enrichment rate was 1.04×10⁵, the control enrichment rate was 9.26×10⁵, and the blank enrichment rate was 2.04×10⁵.

2. Second round of selecting: 5.00×10¹² pfu of the phage-displayed VHH antibody library were used. The target protein was coated on a solid support at a concentration of 5 μg/mL. Before screening, the background from a normal plate and a control plate coated with 5 μg/mL of S protein was subtracted. The unbound phages were washed using 0.3% PBST and then eluted from the solid support using Trypsin. The target enrichment rate was 8.33×10³, the control enrichment rate was 2.50×10⁷, and the blank enrichment rate was 3.13×10⁷.

### 1.2.2 Screening of Monoclonal Antibodies Using Phage ELISA

After the second round of selecting, the eluted phages were used to infect TG1 phage component cells. Individual bacterial clones were randomly selected, and each bacterial clone produced monoclonal phages. Four 96-well were coated with the monoclonal phages. A target protein and a control protein were then added to test the binding specificity of the monoclonal phage to the target protein compared to the control protein. The four 96-well plates were then blocked with skim milk. HRP-conjugated anti-M13 phage antibody was added as a secondary antibody, and then the binding affinity of the monoclonal phages to the target protein were detected and quantified.

Specifically:
1. Coating: A target protein was diluted in a coating buffer with a pH of 9.6; 50 μL of the diluted target protein solution was added to each well of the ELISA plate; and the ELISA plate was then placed in a humidified chamber and incubated at 37° C. for 1 hour, or alternatively, at 4° C. overnight.
2. Blocking: the unbound diluted coating buffer was discarded from the wells; the ELISA plate was inverted onto a clean paper towel and gently tapped to remove any residual liquid; the ELISA plate was washed with PBS; 300 μL of a blocking buffer was added into each well of the ELISA plate; and the ELISA plate was incubated at 37° C. for 1 hour.
3. Primary antibody incubation: the unbound phage solution was discarded from the wells; the ELISA plate was inverted onto a clean paper towel and gently tapped to remove any residual liquid; and the ELISA plate was washed with PBS; HRP-conjugated anti-M13 phage antibody was diluted 1:1000 in 1% Milk-PBS and added (50 μL/well); and the ELISA plate was then incubated at 37° C. for 1 hour.
4. Color development: after incubation, the unbound HRP-conjugated anti-M13 phage antibody was discarded; the ELISA plate was inverted onto a clean paper towel and gently tapped to remove any residual liquid; the ELISA plate was washed four times with PBS; TMB (3,3′,5,5′-tetramethylbenzidine) substrate solution was added (50 μL/well); 2M H₂SO₄ was added to each well to stop the reaction; and absorbance of the stopped reaction was measured at 450 nm using a microplate reader.

### 1.2.3 Sequencing of Monoclonal Clones:

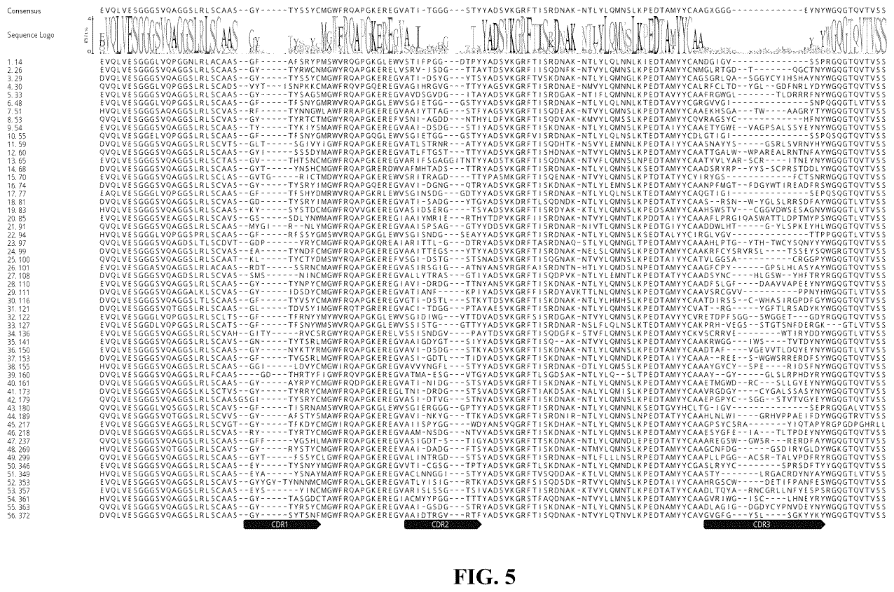

A total of 218 monoclonal phages were selected and sent to a sequencing company for sequence analysis. The sequences obtained were categorized based on the CDR3 region. From the analysis, 56 unique VHH sequences were identified (as shown in FIG. 5). The 56 unique VHH sequences were selected used for subsequent experiments.

### 1.3 In Vitro Expression of Recombinant VHH Antibodies

### 1.3.1 Construction of Fc Fusion Vector and Insertion of VHH Sequences

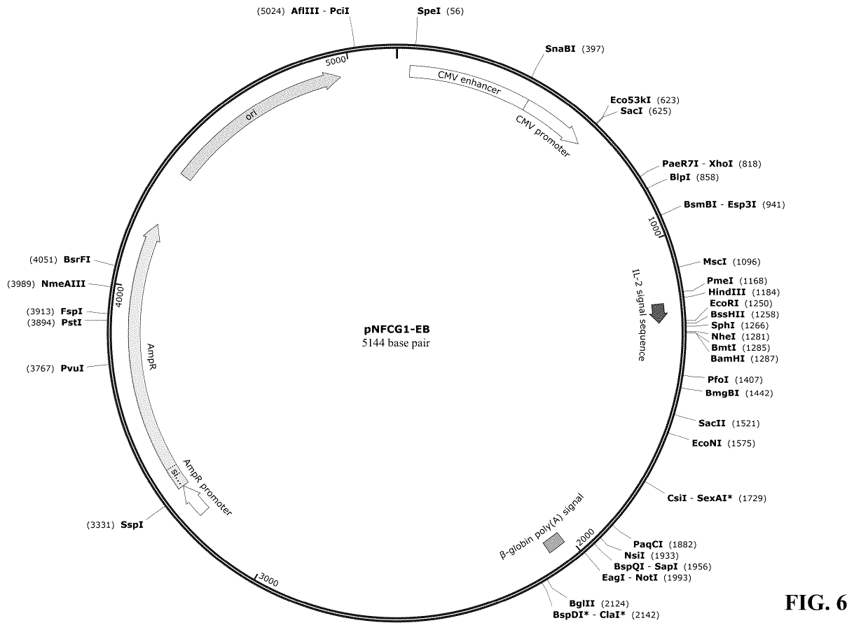

The VHH sequences were inserted into the pNFCG1-EB vector (as shown in FIG. 6) to form a recombinant plasmid.

1.3.2 Transfection of Recombinant Vectors into 293T Cells

Human embryonic kidney cells (293T cells) were collected, resuspended in culture medium to achieve a concentration of 1×10⁶ cells/mL, and incubated at 37° C. with 5% CO₂ to form a cell suspension. 1 μg of the recombinant vector was diluted in 40 μL of KPM medium and mixed; 5 μL of T1 transfection regent (T1:recombinant plasmid=1:5) was diluted in 40 μL of KPM medium, mixed, and incubated at room temperature for 5 minutes; after incubation, the diluted T1 transfection regent was added to the diluted recombinant plasmid solution, mixed, and incubated at room temperature for 30 minutes to form a transfection complex. The transfection complex was added to the cell suspension, mixed, and incubated at 37° C. with 5% CO₂ to form transfected cells. After 72 hours of incubation, the supernatant was collected and analyzed to detect and measure the activity and expression levels of the recombinant VHH antibodies produced by the transfected cells.

### Example 2 Detection of Specificity and Binding Activity of Recombinant VHH Antibodies Against CPV

1. Coating: Antigen P was diluted to a concentration of 5 μg/mL in a carbonate buffer with a pH of 9.6; 100 μL of the diluted antigen P was added to each well of a 96-well ELISA plate; and the 96-well ELISA plate was then incubated overnight at 4° C.

2. Blocking: The unbound coating buffer was discarded from the wells; the 96-well ELISA plate was washed three times with PBST; each well was blocked with 300 μL of a 4% skim milk in PBS; and the 96-well ELISA plate was incubated at 37° C. for 2 hours.

3. Sample addition: The unbound blocking solution was discarded; the 96-well ELISA plate was washed three times with PBST; samples, along with PBS and medium for the control group, were added to the wells (100 μL/well); and the 96-well ELISA plate was then incubated at 37° C. for 1 hour.

4. Secondary antibody incubation: After incubation, the unbound samples were discarded, and the 96-well ELISA plate was washed three times with PBST; the goat anti-human IgG-HRP (Fc) antibody, diluted 1:5000, was added (100 μL/well); and the 96-well ELISA plate was then incubated at 37° C. for 1 hour.

5. Color development: The unbound goat anti-human IgG-RP (Fc) antibody was discarded; the 96-well ELISA plate was washed five times with PBST; TMB substrate solution was added (100 μl/well) and developed in the dark; after color development, 50 μl of 2M hydrochloric acid (HCl) was added to each well to stop the reaction; and the absorbance of the stopped reaction was measured at 450 nm (A450) using a microplate reader

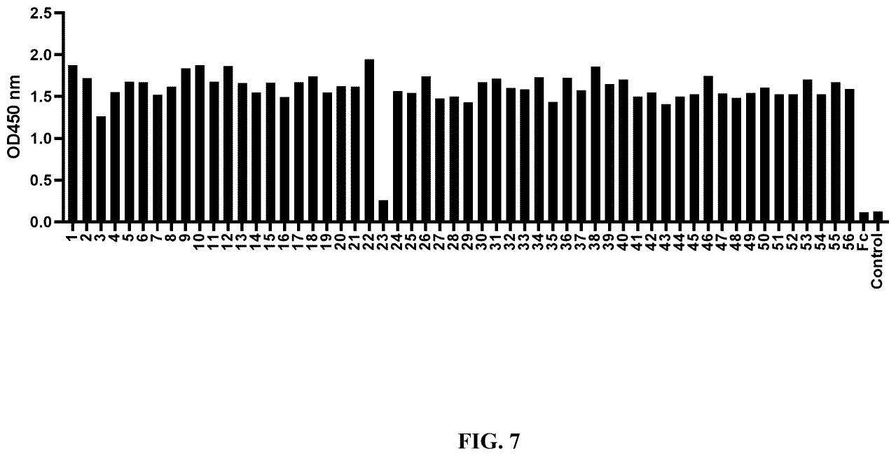

As shown in FIG. 7 and Table 2, the 56 selected VHH antibodies exhibit both binding affinity and specificity compared to the Control group and the control group.

TABLE 2
Binding affinity of 56 VHH antibodies to CPV

| Antibody | |
|---|---|
| number | A450 value |
| 1 | 1.88 |
| 2 | 1.72 |
| 3 | 1.26 |
| 4 | 1.56 |
| 5 | 1.68 |
| 6 | 1.67 |
| 7 | 1.52 |
| 8 | 1.62 |
| 9 | 1.84 |
| 10 | 1.88 |
| 11 | 1.68 |
| 12 | 1.87 |
| 13 | 1.66 |
| 14 | 1.55 |
| 15 | 1.67 |
| 16 | 1.50 |
| 17 | 1.67 |
| 18 | 1.75 |
| 19 | 1.55 |
| 20 | 1.62 |
| 21 | 1.62 |
| 22 | 1.94 |
| 23 | 0.26 |
| 24 | 1.57 |
| 25 | 1.54 |
| 26 | 1.74 |
| 27 | 1.48 |
| 28 | 1.50 |
| 29 | 1.43 |
| 30 | 1.67 |
| 31 | 1.72 |
| 32 | 1.61 |
| 33 | 1.59 |
| 34 | 1.73 |
| 35 | 1.44 |
| 36 | 1.73 |
| 37 | 1.58 |
| 38 | 1.86 |
| 39 | 1.65 |
| 40 | 1.71 |
| 41 | 1.50 |
| 42 | 1.55 |
| 43 | 1.41 |
| 44 | 1.50 |
| 45 | 1.53 |
| 46 | 1.75 |
| 47 | 1.54 |
| 48 | 1.48 |
| 49 | 1.55 |
| 50 | 1.61 |
| 51 | 1.53 |
| 52 | 1.53 |
| 53 | 1.70 |
| 54 | 1.53 |
| 55 | 1.67 |
| 56 | 1.59 |
| Fc | 0.12 |
| Control | 0.13 |

### Example 3 Identification of Neutralizing Activity of VHH Antibodies Against CPV

A neutralization assay was performed as follows: the VHHT antibodies, derived from the final supernatant in Example 1, were prepared at a concentration of 1 μg/mL, with serial dilutions starting from 1:2; each dilution was repeated across four sample wells; the diluted VHHT antibodies were incubated with 100 Tissue Culture Infectious Dose (TCID50) of the CH-HuB-2017 virus at 37° C. for 1 hour. 50 μL of IBRS-2 cells was added to each well of a 96-well plate; the 96-well plate was then incubated at 37° C. with 500 CO₂ for 72 hours; after incubation, the wells were examined using an inverted microscope to observe cytopathic effects; and the neutralizing antibody titers were calculated using the Reed-Muench method.

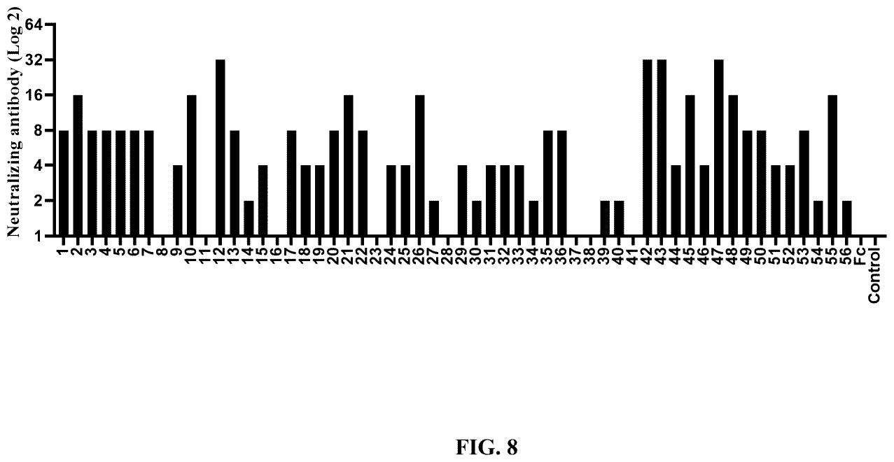

As shown in FIG. 8, out of the 56 VHH antibodies tested for neutralizing activity against CPV, 8 VHH antibodies, specifically antibodies 8, 11, 16, 23, 28, 37, 38, and 41, did not exhibit any neutralizing activity. The remaining 48 VHH antibodies demonstrated varying levels of neutralizing activity. Among the 48 VHHT antibodies, antibodies 2, 10, 45, 48, and 55 showed a neutralizing activity with a titer of ≥16 Furthermore, antibodies 12, 42, 43, and 47 exhibited even higher neutralizing activity, with a titer of: 32:1.

TABLE 3
Neutralizing activity of 56 VHH antibodies against CPV

| Antibody | Neutralizing |
|---|---|
| number | activity |
| 1 | 8:1 |
| 2 | 16:1 |
| 3 | 8:1 |
| 4 | 8:1 |
| 5 | 8:1 |
| 6 | 8:1 |
| 7 | 8:1 |
| 8 | 0 |
| 9 | 4:1 |
| 10 | 16:1 |
| 11 | 0 |
| 12 | 32:1 |
| 13 | 8:1 |
| 14 | 2:1 |
| 15 | 4:1 |
| 16 | 0 |
| 17 | 8:1 |
| 18 | 4:1 |
| 19 | 4:1 |
| 20 | 8:1 |
| 21 | 16:1 |
| 22 | 8:1 |
| 23 | 0 |
| 24 | 4:1 |
| 25 | 4:1 |
| 26 | 16:1 |
| 27 | 2:1 |
| 28 | 0 |
| 29 | 4:1 |
| 30 | 2:1 |
| 31 | 4:1 |
| 32 | 4:1 |
| 33 | 4:1 |
| 34 | 2:1 |
| 35 | 8:1 |
| 36 | 8:1 |
| 37 | 0 |
| 38 | 0 |
| 39 | 2:1 |
| 40 | 2:1 |
| 41 | 0 |
| 42 | 32:1 |
| 43 | 32:1 |
| 44 | 4:1 |
| 45 | 16:1 |
| 46 | 4:1 |
| 47 | 32:1 |
| 48 | 16:1 |
| 49 | 8:1 |
| 50 | 8:1 |
| 51 | 4:1 |
| 52 | 4:1 |
| 53 | 8:1 |
| 54 | 2:1 |
| 55 | 16:1 |
| 56 | 2:1 |
| Fc | 0 |
| Control | 0 |

TABLE 4
Sequences of 48 VHH antibodies with
neutralizing activity against CPV

| Antibody | SEQ ID NO | CDR-1 | SEQ ID NO | CDR-2 | SEQ ID NO | CDR-3 |
|---|---|---|---|---|---|---|
| 1 | SEQID | GFAFSRYPM | SEQ ID | STIFPGGDT | SEQ ID | DGIGVSSP |
| | NO: 1 | | NO: 49 | PY | NO: 97 | |
| 2 | SEQID | GYTYRWCN | SEQ ID | SRVISDGTT | SEQ ID | GLRTGDTQ |
| | NO: 2 | M | NO: 50 | A | NO: 98 | GCTNY |
| 3 | SEQID | GYTYSSYC | SEQ ID | ATIDSYGY | SEQ ID | SGRLQASGG |
| | NO: 3 | M | NO: 51 | TS | NO: 99 | YCYIHSHAY |
| | | | | | | NY |
| 4 | SEQ ID | VYTISNPKK | SEQ ID | AGIHRGVG | SEQ ID | RFCLTDYGL |
| | NO: 4 | CM | NO: 52 | TTY | NO: 100 | GDFNRLYD |
| | | | | | | Y |
| 5 | SEQ ID | GYTYSAGS | SEQ ID | AVDSGVDG | SEQ ID | FRGWGLTL |
| | NO: 5 | M | NO: 53 | ITA | NO: 101 | DRRRFNY |
| 6 | SEQ ID | GFTFSNYGM | SEQ ID | SGIETGGGS | SEQ ID | GVVGISNP |
| | NO: 6 | | NO: 54 | TY | NO: 102 | |
| 7 | SEQ ID | RFTYNNGW | SEQ ID | AAIYTTAG | SEQ ID | EKHSGATW |
| | NO: 7 | L | NO: 55 | STF | NO: 103 | AAGRYTY |
| 9 | SEQ ID | TYTYKIYSM | SEQ ID | AAIDSDGS | SEQ ID | ETYGWEVA |
| | NO: 8 | | NO: 56 | TI | NO: 104 | GPSALSSYE |
| | | | | | | YNY |
| 10 | SEQ ID | GFTFSNYGM | SEQ ID | SGIETGGGS | SEQ ID | GTIGISSP |
| | NO: 9 | | NO: 57 | TY | NO: 105 | |
| 12 | SEQ ID | GYISSSDYM | SEQ ID | ATLFTGSTT | SEQ ID | TTGALWWP |
| | NO: 10 | | NO: 58 | TY | NO: 106 | AREALRNT |
| | | | | | | NFAY |
| 13 | SEQ ID | GVTHTSNC | SEQ ID | ARIFSGAG | SEQ ID | TYVLYARSC |
| | NO: 11 | M | NO: 59 | GITNTY | NO: 107 | RITNEYNY |
| 14 | SEQ ID | GVTHTSNC | SEQ ID | AFMHTADS | SEQ ID | DSRYRPYYS |
| | NO: 12 | M | NO: 60 | TTR | NO: 108 | SCPRSTDDLY |
| 15 | SEQ ID | GVTGRICTM | SEQ ID | SRITRAGDT | SEQ ID | RYGSFCTSN |
| | NO: 13 | | NO: 61 | T | NO: 109 | R |
| 17 | SEQ ID | GFTFSHYDM | SEQ ID | SGINSDGG | SEQ ID | GTIGISEP |
| | NO: 14 | | NO: 62 | DTY | NO: 110 | |
| 18 | SEQ ID | GDTYSRYIM | SEQ ID | ATISADGY | SEQ ID | SRSNWYGL |
| | NO: 15 | | NO: 63 | TG | NO: 111 | SLRRSDFAY |
| 19 | SEQ ID | KYSYSTDC | SEQ ID | ASIDSERGT | SEQ ID | HSWSTVCG |
| | NO: 16 | M | NO: 64 | S | NO: 112 | GVDWSESA |
| | | | | | | GNV |
| 20 | SEQ ID | GSSDLYNW | SEQ ID | AAIYMRIE | SEQ ID | AFLPRGIQA |
| | NO: 17 | M | NO: 65 | RTH | NO: 113 | SWATTLDPT |
| | | | | | | MYPS |
| 21 | SEQ ID | MYGIRNLY | SEQ ID | AAISPSAGG | SEQ ID | DDWLHTGY |
| | NO: 18 | M | NO: 66 | TY | NO: 114 | LSPKEYHL |
| 22 | SEQ ID | GFRFSSYGM | SEQ ID | SGISNDGSE | SEQ ID | GLVGVTTP |
| | NO: 19 | | NO: 67 | AY | NO: 115 | |
| 24 | SEQ ID | EATYNDFC | SEQ ID | AAITTEGSY | SEQ ID | KRFCYSRVR |
| | NO: 20 | M | NO: 68 | TY | NO: 116 | SLTSSEYSQ |
| 25 | SEQ ID | KLTYCTYD | SEQ ID | SGIDSTGST | SEQ ID | VLGGSACR |
| | NO: 21 | M | NO: 69 | S | NO: 117 | GGPY |
| 26 | SEQ ID | RDTSSRNCM | SEQ ID | ASIRSGIGA | SEQ ID | GFCPYGPSL |
| | NO: 22 | | NO: 70 | TN | NO: 118 | HLASYAY |
| 27 | SEQ ID | SMSNINCM | SEQ ID | ALLYTRAS | SEQ ID | DSYNCHLGS |
| | NO: 23 | | NO: 71 | GTI | NO: 119 | WYHFTRY |
| 29 | SEQ ID | GYIDSDYCM | SEQ ID | ATIANFKPI | SEQ ID | VSRCGVVPP |
| | NO: 24 | | NO: 72 | | NO: 120 | NYH |
| 30 | SEQ ID | GFTYVSYC | SEQ ID | GTIDSTLST | SEQ ID | TDIRSSCWH |
| | NO: 25 | M | NO: 73 | K | NO: 121 | ASIRGPDFG |
| | | | | | | Y |
| 31 | SEQ ID | GLTDVSYIM | SEQ ID | ACITDGGP | SEQ ID | TRGYGFTLR |
| | NO: 26 | | NO: 74 | | | |
| | | | | TA | NO: 122 | SADYKY |
| 32 | SEQ ID | GFTFRNYY | SEQ ID | SGIDIWGV | SEQ ID | ETDPFSGGS |
| | NO: 27 | M | NO: 75 | TTD | NO: 123 | WGGETGDY |
| 33 | SEQ ID | GFTFSNYW | SEQ ID | SSISTGGTT | SEQ ID | PRHVEGSST |
| | NO: 28 | M | NO: 76 | Y | NO: 124 | GTSNFDERG |
| | | | | | | K |
| 34 | SEQ ID | GITYRVCSR | SEQ ID | SSISNDGVP | SEQ ID | SCRRVEWTI |
| | NO: 29 | | NO: 77 | A | NO: 125 | RYDDY |
| 35 | SEQ ID | GNTYTSRL | SEQ ID | AAIGDYGT | SEQ ID | KRWGGIWS |
| | NO: 30 | M | NO: 78 | SIY | NO: 126 | TVTDYNY |
| 36 | SEQ ID | GYNYKTYR | SEQ ID | AVIDSDGS | SEQ ID | DTAFVGEV |
| | NO: 31 | M | NO: 79 | TK | NO: 127 | VTLDQYEY |
| | | | | | | NY |
| 39 | SEQ ID | GDTHRTYFI | SEQ ID | ATMAESGV | SEQ ID | AYGYGLSL |
| | NO: 32 | | NO: 80 | TG | NO: 128 | RPHDYRY |
| 40 | SEQ ID | GYAYRPYC | SEQ ID | ATINIDGST | SEQ ID | ETMGWDRC |
| | NO: 33 | M | NO: 81 | S | NO: 129 | SLLGYEYNY |
| 42 | SEQ ID | SGITYSRYC | SEQ ID | ASIDTVGST | SEQ ID | STVTVGYEY |
| | NO: 34 | M | NO: 82 | N | NO: 130 | EPGPYCSGG |
| 43 | SEQ ID | GFTISRNAM | SEQ ID | SGIERGGG | SEQ ID | GIGVSEP |
| | NO: 35 | | NO: 83 | GPTY | NO: 131 | |
| 44 | SEQ ID | GYAFSTYSM | SEQ ID | AVINKYGT | SEQ ID | HLNLWIGR |
| | NO: 36 | | NO: 84 | TK | NO: 132 | HVPPAEIFD |
| | | | | | | Y |
| 45 | SEQ ID | GYTFKDYC | SEQ ID | AIISPYGGW | SEQ ID | GPSYCSRAY |
| | NO: 37 | M | NO: 85 | D | NO: 133 | IQTAPY |
| 46 | SEQ ID | RYTYRTYC | SEQ ID | AAMNSDG | SEQ ID | ESYGFEIAT |
| | NO: 38 | M | NO: 86 | NTV | NO: 134 | LTPDEYNY |
| 47 | SEQ ID | GFFVGSHLM | SEQ ID | ASIGDTSTI | SEQ ID | AREGSWGW |
| | NO: 39 | | NO: 87 | G | NO: 135 | SRRERDFAY |
| 48 | SEQ ID | GYRYSTYC | SEQ ID | AAIDADGF | SEQ ID | GCNFDGGS |
| | NO: 40 | M | NO: 88 | TS | NO: 136 | DIRYGLDY |
| 49 | SEQ ID | GYRYSTYC | SEQ ID | ALINTRGD | SEQ ID | PLLPGGACS |
| | NO: 41 | M | NO: 89 | STS | NO: 137 | RTALVPDFR |
| | | | | | | Y |
| 50 | SEQ ID | GYTFSSYCL | SEQ ID | VTICGSGTP | SEQ ID | SLRYYCSPR |
| | NO: 42 | | NO: 90 | T | NO: 138 | SDFTY |
| 51 | SEQ ID | EYTYSNYY | SEQ ID | ACLNNGGI | SEQ ID | STYLRGACR |
| | NO: 43 | M | NO: 91 | STY | NO: 139 | DYNYAY |
| 52 | SEQ ID | EYAYSNAY | SEQ ID | ATLYISIGR | SEQ ID | HRGSCWDE |
| | NO: 44 | M | NO: 92 | TK | NO: 140 | TIFPANFES |
| 53 | SEQ ID | GYYGYTYN | SEQ ID | ARISLSSGT | SEQ ID | DLTQYARN |
| | NO: 45 | NNMCM | NO: 93 | SI | NO: 141 | CGRLLNFYE |
| | | | | | | SPS |
| 54 | SEQ ID | EYSYINCM | SEQ ID | ACMYYPG | SEQ ID | GVRIWGISC |
| | NO: 46 | | NO: 94 | GTTT | NO: 142 | LHNEYRY |
| 55 | SEQ ID | GYTYRSYC | SEQ ID | AAIGSDGS | SEQ ID | DLAGIGDGD |
| | NO: 47 | M | NO: 95 | TR | NO: 143 | YCYPNVDE |
| | | | | | | YNY |
| 56 | SEQ ID | GYSYTSNFM | SEQ ID | AAIDTRGV | SEQ ID | GVGFGYSLS |
| | NO: 48 | | NO: 96 | RTF | NO: 144 | GKYYKY |

It will be obvious to those skilled in the art that changes and modifications may be made, and therefore, the aim in the appended claims is to cover all such changes and modifications.

What is claimed is:

1. A single-domain antibody or an antigen-binding fragment thereof for neutralizing canine parvovirus (CPV), the single-domain antibody or antigen-binding fragment thereof comprising a heavy chain encompassing a variable region, and the variable region comprising three complementarity-determining regions (CDRs): CDR1, CDR2, and CDR3; the three complementarity-determining regions CDR1, CDR2, and CDR3 having a combination of amino acid sequences as follows:
(1) CDR1 shown in SEQ ID NO: 1, CDR2 shown in SEQ ID NO: 49, and CDR3 shown in SEQ ID NO: 97;
(2) CDR1 shown in SEQ ID NO: 2, CDR2 shown in SEQ ID NO: 50, and CDR3 shown in SEQ ID NO: 98;
(3) CDR1 shown in SEQ ID NO: 3, CDR2 shown in SEQ ID NO: 51, and CDR3 shown in SEQ ID NO: 99;
(4) CDR1 shown in SEQ ID NO: 4, CDR2 shown in SEQ ID NO: 52, and CDR3 shown in SEQ ID NO: 100;
(5) CDR1 shown in SEQ ID NO: 5, CDR2 shown in SEQ ID NO: 53, and CDR3 shown in SEQ ID NO: 101;
(6) CDR1 shown in SEQ ID NO: 6, CDR2 shown in SEQ ID NO: 54, and CDR3 shown in SEQ ID NO: 102;
(7) CDR1 shown in SEQ ID NO: 7, CDR2 shown in SEQ ID NO: 55, and CDR3 shown in SEQ ID NO: 103;
(9) CDR1 shown in SEQ ID NO: 8, CDR2 shown in SEQ ID NO: 56, and CDR3 shown in SEQ ID NO: 104;
(10) CDR1 shown in SEQ ID NO: 9, CDR2 shown in SEQ ID NO: 57, and CDR3 shown in SEQ ID NO: 105;
(12) CDR1 shown in SEQ ID NO: 10, CDR2 shown in SEQ ID NO: 58, and CDR3 shown in SEQ ID NO: 106;
(13) CDR1 shown in SEQ ID NO: 11, CDR2 shown in SEQ ID NO: 59, and CDR3 shown in SEQ ID NO: 107;
(14) CDR1 shown in SEQ ID NO: 12, CDR2 shown in SEQ ID NO: 60, and CDR3 shown in SEQ ID NO: 108;
(15) CDR1 shown in SEQ ID NO: 13, CDR2 shown in SEQ ID NO: 61, and CDR3 shown in SEQ ID NO: 109;
(17) CDR1 shown in SEQ ID NO: 14, CDR2 shown in SEQ ID NO: 62, and CDR3 shown in SEQ ID NO: 110;
(18) CDR1 shown in SEQ ID NO: 15, CDR2 shown in SEQ ID NO: 63, and CDR3 shown in SEQ ID NO: 111;
(19) CDR1 shown in SEQ ID NO: 16, CDR2 shown in SEQ ID NO: 64, and CDR3 shown in SEQ ID NO: 112;
(20) CDR1 shown in SEQ ID NO: 17, CDR2 shown in SEQ ID NO: 65, and CDR3 shown in SEQ ID NO: 113;
(21) CDR1 shown in SEQ ID NO: 18, CDR2 shown in SEQ ID NO: 66, and CDR3 shown in SEQ ID NO: 114;
(22) CDR1 shown in SEQ ID NO: 19, CDR2 shown in SEQ ID NO: 67, and CDR3 shown in SEQ ID NO: 115;
(24) CDR1 shown in SEQ ID NO: 20, CDR2 shown in SEQ ID NO: 68, and CDR3 shown in SEQ ID NO: 116;
(25) CDR1 shown in SEQ ID NO: 21, CDR2 shown in SEQ ID NO: 69, and CDR3 shown in SEQ ID NO: 117;
(26) CDR1 shown in SEQ ID NO: 22, CDR2 shown in SEQ ID NO: 70, and CDR3 shown in SEQ ID NO: 118;
(27) CDR1 shown in SEQ ID NO: 23, CDR2 shown in SEQ ID NO: 71, and CDR3 shown in SEQ ID NO: 119;
(29) CDR1 shown in SEQ ID NO: 24, CDR2 shown in SEQ ID NO: 72, and CDR3 shown in SEQ ID NO: 120;
(30) CDR1 shown in SEQ ID NO: 25, CDR2 shown in SEQ ID NO: 73, and CDR3 shown in SEQ ID NO: 121;
(31) CDR1 shown in SEQ ID NO: 26, CDR2 shown in SEQ ID NO: 74, and CDR3 shown in SEQ ID NO: 122;
(32) CDR1 shown in SEQ ID NO: 27, CDR2 shown in SEQ ID NO: 75, and CDR3 shown in SEQ ID NO: 123;
(33) CDR1 shown in SEQ ID NO: 28, CDR2 shown in SEQ ID NO: 76, and CDR3 shown in SEQ ID NO: 124;
(34) CDR1 shown in SEQ ID NO: 29, CDR2 shown in SEQ ID NO: 77, and CDR3 shown in SEQ ID NO: 125;
(35) CDR1 shown in SEQ ID NO: 30, CDR2 shown in SEQ ID NO: 78, and CDR3 shown in SEQ ID NO: 126;
(36) CDR1 shown in SEQ ID NO: 31, CDR2 shown in SEQ ID NO: 79, and CDR3 shown in SEQ ID NO: 127;
(39) CDR1 shown in SEQ ID NO: 32, CDR2 shown in SEQ ID NO: 80, and CDR3 shown in SEQ ID NO: 128;
(40) CDR1 shown in SEQ ID NO: 33, CDR2 shown in SEQ ID NO: 81, and CDR3 shown in SEQ ID NO: 129;
(42) CDR1 shown in SEQ ID NO: 34, CDR2 shown in SEQ ID NO: 82, and CDR3 shown in SEQ ID NO: 130;
(43) CDR1 shown in SEQ ID NO: 35, CDR2 shown in SEQ ID NO: 83, and CDR3 shown in SEQ ID NO: 131;
(44) CDR1 shown in SEQ ID NO: 36, CDR2 shown in SEQ ID NO: 84, and CDR3 shown in SEQ ID NO: 132;
(45) CDR1 shown in SEQ ID NO: 37, CDR2 shown in SEQ ID NO: 85, and CDR3 shown in SEQ ID NO: 133;
(46) CDR1 shown in SEQ ID NO: 38, CDR2 shown in SEQ ID NO: 86, and CDR3 shown in SEQ ID NO: 134;
(47) CDR1 shown in SEQ ID NO: 39, CDR2 shown in SEQ ID NO: 87, and CDR3 shown in SEQ ID NO: 135;
(48) CDR1 shown in SEQ ID NO: 40, CDR2 shown in SEQ ID NO: 88, and CDR3 shown in SEQ ID NO: 136;
(49) CDR1 shown in SEQ ID NO: 41, CDR2 shown in SEQ ID NO: 89, and CDR3 shown in SEQ ID NO: 137;
(50) CDR1 shown in SEQ ID NO: 42, CDR2 shown in SEQ ID NO: 90, and CDR3 shown in SEQ ID NO: 138;
(51) CDR1 shown in SEQ ID NO: 43, CDR2 shown in SEQ ID NO: 91, and CDR3 shown in SEQ ID NO: 139;
(52) CDR1 shown in SEQ ID NO: 44, CDR2 shown in SEQ ID NO: 92, and CDR3 shown in SEQ ID NO: 140;
(53) CDR1 shown in SEQ ID NO: 45, CDR2 shown in SEQ ID NO: 93, and CDR3 shown in SEQ ID NO: 141;
(54) CDR1 shown in SEQ ID NO: 46, CDR2 shown in SEQ ID NO: 94, and CDR3 shown in SEQ ID NO: 142;
(55) CDR1 shown in SEQ ID NO: 47, CDR2 shown in SEQ ID NO: 95, and CDR3 shown in SEQ ID NO: 143; or
(56) CDR1 shown in SEQ ID NO: 48, CDR2 shown in SEQ ID NO: 96, and CDR3 shown in SEQ ID NO: 144; and
CDR1 has a sequence represented by one of SEQ ID NO: 1 to SEQ ID NO: 48, an amino acid sequence having at least 80%, 85%, 90%, 91%, 92%, 93%, 94%, 95%, 96%, 97%, 98%, or 99% sequence identity to one of SEQ ID NO: 1 to SEQ ID NO: 48, or a sequence variant having no more than 8 amino acid substitutions from one of SEQ ID NO: 1 to SEQ ID NO: 48; CDR2 has a sequence represented by one of SEQ ID NO: 49 to SEQ ID NO: 96, an amino acid sequence having at least 80%, 85%, 90%, 91%, 92%, 93%, 94%, 95%, 96%, 97%, 98%, or 99% sequence identity to one of SEQ ID NO: 49 to SEQ ID NO: 96, or a sequence variant having no more than 8 amino acid substitutions from one of SEQ ID NO: 49 to SEQ ID NO: 96; and CDR3 has a sequence represented by one of SEQ ID NO: 97 to SEQ ID NO: 144, an amino acid sequence having at least 80%, 85%, 90%, 91%, 92%, 93%, 94%, 95%, 96%, 97%, 98%, or 99% sequence identity to one of SEQ ID NO: 97 to SEQ ID NO: 144, or a sequence variant having no more than 8 amino acid substitutions from one of SEQ ID NO: 97 to SEQ ID NO: 144.

2. The single-domain antibody or an antigen-binding fragment thereof of claim 1, being selected from Fab, Fab′, F(ab′)2, Fv, single-chain Fv (scFv), single-chain Fab, diabody, single-domain antibody or sdAb (nanobody), camelid Ig, IgNAR, F(ab)′3 fragment, bispecific scFv, (scFv)2, mini antibody, bispecific antibody, trispecific antibody, tetraspecific antibody, and disulfide-stabilized Fv protein (dsFv).

3. The single-domain antibody or an antigen-binding fragment thereof of claim 1, further comprising a constant region selected from porcine IgG, human IgG, and chicken IgY.

4. The single-domain antibody or an antigen-binding fragment thereof of claim 3, wherein the constant region IgG is IgG1, IgG2a, IgG2b, and/or IgG4.

5. The single-domain antibody or an antigen-binding fragment thereof of claim 1, wherein the CPV is isolated from CPV-2a strain.

6. A nucleic acid molecule encoding the single-domain antibody or an antigen-binding fragment thereof of claim 1.

7. A recombinant vector or host cell comprising the nucleic acid molecule of claim 6.

8. The recombinant vector or host cell of claim 7, wherein the recombinant vector is selected from DNA, RNA, viral vectors, transposons, or a combination thereof.

9. The recombinant vector or host cell of claim 8, wherein the viral vectors comprise lentivirus, adenovirus, adeno-associated virus (AAV), retrovirus, or a combination thereof.

10. A chimeric antibody comprising the single-domain antibody or antigen-binding fragment thereof of claim 1.

11. A fusion or conjugate comprising the single-domain antibody or antigen-binding fragment thereof of claim 1 and a heterologous molecule to which the fusion or conjugate is attached.

12. The fusion or conjugate of claim 11, wherein the heterologous molecule is proteins, peptides, markers, drugs, or cytotoxic agents.

13. A composition comprising the single-domain antibody or antigen-binding fragment thereof of claim 1, a nucleic acid molecule encoding the single-domain antibody or antigen-binding fragment thereof, a recombinant vector or host cell comprising the single-domain antibody or antigen-binding fragment thereof, a chimeric antibody comprising the single-domain antibody or antigen-binding fragment thereof, and/or a fusion or conjugate comprising the single-domain antibody or antigen-binding fragment thereof.

14. A method for diagnosis, prevention, and/or treatment of viral infections comprising administering a patient in need thereof the single-domain antibody or antigen-binding fragment thereof of claim 1, a nucleic acid molecule encoding the single-domain antibody or antigen-binding fragment thereof, a recombinant vector or host cell comprising the single-domain antibody or antigen-binding fragment thereof, a chimeric antibody comprising the single-domain antibody or antigen-binding fragment thereof, and/or a fusion or conjugate comprising the single-domain antibody or antigen-binding fragment thereof.

15. A kit for detecting CPV comprising an instruction manual and a reagent for detecting the single-domain antibody or antigen-binding fragment thereof of claim 1, a nucleic acid molecule encoding the single-domain antibody or antigen-binding fragment thereof, a recombinant vector or host cell comprising the single-domain antibody or antigen-binding fragment thereof, a chimeric antibody comprising the single-domain antibody or antigen-binding fragment thereof, and/or a fusion or conjugate comprising the single-domain antibody or antigen-binding fragment thereof.
